(19) United States
(12) Patent Application Publication
(10) Pub. No.: US 2009/0306025 A1
(43) Pub. Date: Dec. 10, 2009

(54) Method and composition for skin inflammation and discoloration

(75) Inventor: Edward M. Lane, Weston, CT (US)

Correspondence Address: ROTHWELL, FIGG, ERNST & MANBECK, P.C., 1425 K STREET, N.W., SUITE 800, WASHINGTON, DC, 20005, US

(73) Assignee: Fairfield Clinical Trials, LLC, Bridgeport, CT (US)

(21) Appl. No.: 12/457,116

(22) Filed: Jun. 1, 2009

Related U.S. Application Data
(60) Provisional application No. 61/057,700, filed on May 30, 2008.
(60) Provisional application No. 61/088,440, filed on Aug. 13, 2008.
(60) Provisional application No. 61/118,191, filed on Nov. 26, 2008.
(60) Provisional application No. 61/159,984, filed on Mar. 13, 2009.

(51) Int. Cl. A61K 31/60; A61K 31/445; A61P 11/02

(52) U.S. Cl. 514/161; 514/317

(57) ABSTRACT

The invention provides a method and compound for treating darkness and/or swelling/inflammation of the skin of humans. An antihistamine compound and a non-steroidal anti-inflammatory drug (NSAID) compound in combination have been found to effectively treat under eye darkness, swelling and puffiness in particular, when applied topically to the affected skin.

### CROSS REFERENCE TO APPLICATIONS

This application claims benefit of U.S. Provisional Patent Application Ser. No. 61/057,700, filed May 30, 2008; Ser. No. 61/088,440, filed Aug. 13, 2008; Ser. No. 61/118,191, filed Nov. 26, 2008; and Ser. No. 61/159,984, filed Mar. 13, 2009. The disclosures of each of these provisional applications is incorporated by reference herein in its entirety.

### BACKGROUND OF THE INVENTION

1. Technical Field

This invention generally relates to the field of medical dermatology, allergy and cosmetics. This application describes a topically applied medical treatment composition and methods, which provide improvement in the cosmetic appearance of the dark circles and/or swelling/inflammation that can occur beneath the eyes of humans.

2. Description of the Background Art

Most people will develop a darkening of the skin below or around their eyes at various times in their lives. These dark areas (also known as dark circles or “allergic shiners” and referred to as such herein) may be circular in shape or any other shape. Common causes include but are not limited to persistent eye rubbing, sleep disorders, allergies, allergic and non-allergic (perennial) rhinitis, hay fever, eczema, pallor, aging, dehydration, and trauma. Periods of appearance of the darkening may come and go, but for some people, mainly women, these darkened areas can remain a relatively constant feature.

The dark circles that appear under the eyes are thought to represent vasodilation and engorgement of the veins in the soft tissues beneath the eyes, and extravasation of blood, blood pigments and blood products into the subcutaneous soft tissues. Because of the thin skin in this area, the engorged veins can be visible as a discolored area. In addition, there can be swelling of the soft tissues beneath the eyes, due to increased permeability of post-capillary venules or peri-orbital fat herniation.

Swelling of the skin below the eyes or puffiness around the eye area can occur independently or concurrently with dark circles. Common causes of the swelling can include but are not limited to aging, with and without peri-orbital fat herniation, persistent eye rubbing, sleep disorders, allergies, allergic and non-allergic (perennial) rhinitis, hay fever, eczema, pallor, dehydration, drug reactions, and trauma. Although the degree of swelling around the eyes comes and goes, once it occurs, it rarely resolves spontaneously.

Although under-eye darkness and swelling do not cause specific morbidity, they are a source for cosmetic concern. Pharmacies, health food stores, doctors, offices and the internet propose lotions, creams, and other compositions which allege improvement in the cosmetic appearance of dark circles and under eye swelling. Most contain a vasoconstricting agent (such as pseudoephedrine and/or caffeine) to shrink the underlying blood vessels thought to cause the dark circles. Other agents are designed to absorb blood and blood products which extravasate from capillaries into the under-eye tissues. These agents are of limited effect and, even when applied topically, may produce some systemic effects, acting as neurotransmitters which can adversely affect cardiac rhythm and/or blood pressure. Depending on the degree of systemic absorption, there may be other effects, such as neurologic effects, as well. This is a major drawback to the prior art compositions. Accordingly, there is a great need in the art for a composition and method that is effective in treating darkness and puffiness under and around the eye, and that lacks the negative features of previous treatments. In particular, there is a need in the art for a treatment that diminishes the dark circles and/or under eye swelling yet has no effect on cardiac rhythm, blood pressure, or other systemic effects. Such treatments also preferably would benefit or treat other dermatoses (redness, swelling or inflammation of the skin of the face or body).

### SUMMARY OF THE INVENTION

Therefore, embodiments of the invention provide a topical composition which comprises an antihistamine compound, a non-steroidal anti-inflammatory drug (NSAID) compound and a pharmaceutically acceptable vehicle for topical administration. Preferred embodiments relate to such topical compositions wherein the antihistamine compound is selected from the group consisting of fexofenadine, loratadine, desloratadine, azelastine, cetirizine and levocetirizine, and most preferably fexofenadine. Topical compositions of preferred embodiments contain about 0.0001% to about 99% of the antihistamine compound by weight, or about 0.0001% to about 50%, about 0.001% to about 10%, about 0.01% to about 5%, about 0.1% to about 3%, about 0.5% to about 2%, or about 1% of the antihistamine compound by weight.

Preferred topical compositions contain an NSAID compound selected from the group consisting of ibuprofen, aspirin, ampyrone, celecoxib, diclofenac, diflunisal, droxcam, indomethacin, licofelone, mefanamic acid, naproxen, nimesulide, phenylbutazone, proicam, rofecoxib, valdecoxib, omega-3 fatty acids, and any combination thereof. Topical compositions wherein the NSAID compound is ibuprofen are most preferred. Topical compositions of preferred embodiments contain about 0.0001% to about 99% of the NSAID compound by weight, or about 0.0001% to about 50%, about 0.001% to about 10%, about 0.01% to about 5%, about 0.1% to about 3%, about 0.5% to about 2%, or about 1% of the NSAID compound by weight.

The most preferred compositions contain fexofenadine and ibuprofen.

Embodiments of the invention also include methods of treating swelling, puffiness, redness, darkness or inflammation of skin of a human in need thereof, which comprises topically applying to the skin the topical compositions described above, including to any affected skin and to the eye area.

Such methods generally involve applying about 0.0001 cc to about 1 cc of the topical composition per 1-2 or 1-10 square inch skin area, or about 0.0001 cc to about 1 cc, about 0.001 cc to about 0.5 cc, about 0.001 cc to about 0.5 cc, about 0.01 cc to about 0.3 cc, about 0.01 cc to about 0.3 cc, about 0.1 cc to about 0.2 cc, or about 0.1 cc to about 0.2 cc of the topical composition to the same skin area.

One embodiment in particular relates to a method of treating darkness of the skin under or around the eye of a human in need thereof, which comprises topically applying to the affected skin a topical composition as described herein.

### DETAILED DESCRIPTION OF THE PREFERRED EMBODIMENTS

Histamine, (2-(4-imidazolyl)-ethyl-amine; 4-aminoethylglyoxaline), is a dibasic vasoactive amine that is located in most body tissues, but is highly concentrated in the lungs, skin, and gastrointestinal tract. Histamine is stored in mast cells and basophils. Ionic forces within intracellular granules hold histamine by macroheparin. The interaction between allergen and IgE, bound to the surface of mast cells and basophils by a surface receptor that binds the Fc fragment of IgE, leads to degranulation of these cells, with release of mediators, such as histamine, leukotrienes, substance P, interleukins, bradykinins and kallikreins, among others. Histamine's main physiological actions include stimulation of gastric secretion, contraction of most smooth muscle, cardiac stimulation, vasodilatation, and increased vascular permeability. when injected intradermally, histamine causes vasodilatation, wheal, and flare. Vasodilatation of small arterioles and precapillary sphincters causes reddening, while increased permeability of post capillary veins causes the wheal. Histamine also induces the release of a vasodilating mediator, thus producing the flare.

Histamine induces endothelial cells to synthesize vascular smooth muscle relaxants, including prostaglandin and nitric oxide, which cause vasodilatation. Histamine increases capillary permeability. Increased vascular permeability causes fluid to escape from capillaries into the tissues, which leads to the classic symptoms of an allergic reaction, a runny nose and watery eyes. It is thought to be the major mediator of the acute inflammatory response, although histamine Hl antagonists have little effect on acute inflammation. Histamine produces many of the effects of inflammation and hypersensitivity, including vasodilatation, edema, increased vascular permeability, and smooth muscle contraction. Acting on H2 receptors, histamine increases heart rate and cardiac output and stimulates gastric acid secretion.

In 1937, Bovet and Staub discovered the first H1 receptor antagonist. Antihistamines suppress the histamine-induced wheal and flare response by blocking the binding of histamine to its receptors on nerves, vascular smooth muscle, glandular cells, endothelium, and mast cells. They effectively exert competitive antagonism of histamine for H1 receptors. Although widespread in use, major central nervous system adverse effects such as sedation and performance deficits, and their anticholinergic activities, have introduced problems with their widespread usefulness.

Antihistamines are a broad class of pharmacologic agents that include first generation, centrally acting H1-receptor antagonists (such as diphenhydramine) and the newer, second generation, non-sedating H1 blockers (e.g. fexofenadine, loratidine, desloratidine, azelastine, cetirizine, and levocetirizine). Other antihistaminic agents, such as cimetidine, work primarily at H2 receptors, causing inhibition of gastric secretion. Other experimental antihistamines act on presynaptic H3 and H4 receptors.

Second generation antihistamines, such as fexofenadine, loratidine, desloratidine, azelastine, cetirizine, and levocetirizine, among others, are peripherally selective H1 receptor antagonists. They bind much more selectively to peripheral H1 receptors and have a lower binding affinity for the cholinergic and alpha-adrenergic receptor sites than first generation antihistamines. In addition, they are lipophobic and therefore do not pass easily across the blood-brain barrier, thus causing much less sedation. These second generation antihistamines are popular for treatment of allergic reactions because their specificity for the peripheral histamine receptor site reduces or eliminates many adverse side effects. Systemic antihistamines do not improve under eye dark circles when taken systemically (orally) or when used alone topically.

The majority of NSAID compounds are believed to work through inhibition of cyclo-oxygenase (COX), thus inhibiting prostaglandin synthesis. Ibuprofen, 2-[4-(2-methylpropyl) phenyl] propanoic acid, is a non-selective non-steroidal anti-inflammatory drug (NSAID) that also exhibits analgesic and antipyretic properties. Ibuprofen is used orally for anti-inflammatory and analgesic effects in the symptomatic treatment of mild to moderate pain or fever.

Ibuprofen inhibits both cyclo-oxygenase-1 (COX-1) and cyclo-oxygenase-2 (COX-2), although it is believed that Ibuprofen's analgesic, antipyretic, and anti-inflammatory activities are achieved principally through COX-2 inhibition. COX-1 inhibition is believed to be responsible for Ibuprofen's unwanted side effects on platelet aggregation and on the gastro-intestinal mucosa (e.g. gastritis, ulceration, and/or bleeding). Other NSAID compositions that act at the COX enzymes include, for example ibuprofen, aspirin, ampyrone, celecoxib, diclofenac, diflunisal, droxcam, indomethacin, licofelone, mefanamic acid, naproxen, nimesulide, phenylbutazone, proicam, rofecoxib, valdecoxib, omega-3 fatty acids, and any combination thereof. Any NSAID or other compound that inhibits COX-1, COX-2, COX-3 or any combination thereof, and in general anti-inflammatory compounds, are contemplated for use as part of this invention, although non-specific NSAID compounds and COX-2 inhibitors generally are preferred.

Dark areas of the skin under the eyes and accompanying swelling can be caused by vasodilatation and changes in capillary permeability. Swelling beneath the eyes (alone or accompanied by dark areas) can be caused by extravasation of tissue fluid from post-capillary venules or peri-orbital fat herniation. While histamine promotes vasodilatation and capillary permeability, prostaglandins do not. Therefore, NSAID compounds would not be expected to beneficially affect eye area darkness. Systemic NSAIDS and topical monotherapy NSAIDS do not improve dark circles or skin swelling beneath the eyes. Moreover, Ibuprofen tends to elevate blood pressure and would be assumed to increase blood flow to the area and thus not improve dark circles or swelling below the eyes. Certain antihistamine drugs have significant anticholinergic and alpha-adrenergic effects, so that they can cause flushing of the skin, which would not be helpful in reducing eye area darkness or inflammation.

Nonetheless, a combination of one or more antihistamine and an anti-inflammatory drug compound, such as an NSAID, surprisingly promotes improvement in the appearance of dark circles and the swelling that occurs beneath the eyes when applied topically. Any H1, H2, H3, H4 (or any combination thereof) antihistamine composition is contemplated for use in this invention, although H1 histamine antagonists or blockers (antihistamines) are preferred. The so-called “second generation” H1 antihistamines are most preferred.

Antihistamine compounds that are useful in the inventive compositions include any H1 receptor inhibiting compound, therefore the term “antihistamine” as used herein, includes any such compound. Specific examples of the “first generation” H1 antihistamines include but are not limited to acrivastine, brompheniramine, chlorpheniramine, clemastine, diphenhydramine, doxylamine, hydroxyzine, pheniramine, and promethazine. Specific examples of the preferred “second generation” H1 antihistamines include, but are not limited to azelastine, cetirizine, desloratidine, fexofenadine, levocetirizine, loratidine, and olopatadine. Useful compounds may be members of any of the 7 structural classes of antihistamine (alkylamines, ethanolamines, ethylenediamines, phenothiazine, piperidines, piperazines or norpiperidine imidazoazepines). Preferred antihistamine compounds are fexofenadine, loratadine, desloratadine, azelastine, cetirizine and levocetirizine.

NSAID compounds that are contemplated for use with the invention include salicylates, arylalkanoic acid NSAIDS, arylpripionic acid NSAIDS, N-arylanthranilic (fenamic) acid NSAIDs, pyrazolidine derivative NSAIDS, oxicam NSAID compounds, selective COX-2 inhibitors, sulphonamilide NSAID compounds, and other compounds such as 5-LOX/COX inhibitors. The term NSAID, as used herein, therefore refers to any non-steroidal anti-inflammatory drug or COX (COX-1, COX-2 or COX-3) inhibitor compound. Non-limiting examples of suitable compounds are acetylsalicylic acid (aspirin), ampyrone, celecoxib, diclofenac, diflunisal, droxcam, ibuprofen, indomethacin, licofelone, mefanamic acid, naproxen, nimesulide, omega-3 fatty acids, phenylbutazone, proicam, rofecoxib, valdecoxib or any combination thereof.

In addition to non-steroidal anti-inflammatory compounds, steroid anti-inflammatory compounds and/or leukotriene blockers optionally may be included in inventive compositions. For example, steroids (including glucocorticoids, mineral corticoids and adrenal-corticoids) include, but are not limited to, cortisone, hydrocortisone, prednisone, prednisilone, triamcinolone, beclomethasone, ciclesonide, methylprednisilone, betamethasone, fludrocortisone, DOCA, aldosterone, estrogen, androgen, and progesterone. Suitable leukotriene blockers include but are not limited to monoleukast, zileuton and zafirlukast. Other compounds which also may be used in the inventive compositions are vitamin K, vitamin C, grape seed oil, caffeine, pseudoephedrine, ephedrine, topical bleaching agents, steroids, alkanolamines, Hylexin®, retinol, and tetrapeptides.

Second generation antihistamines have less anticholinergic and alpha-adrenergic effect than first generation antihistamines, and cause less vasodilatation and capillary permeability. Therefore, these compounds are preferred. Ibuprofen has both COX-1 and COX-2 inhibition which prevents prostaglandin synthesis, lessening the occurrence of vasodilation and of capillary permeability. Ibuprofen also increases arteriole tone, which can result in a blood pressure rise. The decreased alpha-adrenergic effect of second generation antihistamines lessens the rise in blood pressure caused by ibuprofen, resulting in less blood flow to the area and less venous engorgement. Without wishing to be bound by theory, these factors are believed to result in a superior effect on under-eye darkness and swelling when an antihistamine and an NSAID are used in combination. The preferred inventive products therefore combine an antihistamine, preferably a second generation antihistamine, together with an NSAID, preferably ibuprofen. The vehicle preferably is water-soluble or is an emulsion and is compatible with the skin of the eye area. Preferably, the vehicle has been clinically tested and does not cause eye or skin irritation.

The inventive compositions also may optionally contain additional ingredients that can promote soothing of the skin or health of the skin, including ingredients that promote a youthful appearance. Such optional ingredients can include botanical extracts such as aloe or green tea, vitamins and antioxidants such as vitamin C, vitamin A or retinol, vitamin E, vitamin K, astringents, hyaluronic acid, omega-3 fatty acids, sunscreen, grape seed oil, caffeine, pseudoephedrine, ephedrine, topical bleaching agents, alkanolamines, Hylexin® and/or tetrapeptides. The compositions also can include a steroid and/or a leukotriene blocker. Additionally, ingredients that aid in penetration of the active ingredients into and/or through the skin also optionally may be included in the inventive compositions.

The vehicle or carrier for the antihistamine and NSAID or other optional active compounds may be any excipient or combination of excipients which are suitable for topical delivery of the active compounds to the skin of the face or body wherever irritation, redness, inflammation or darkness has occurred, and particularly to the skin around the eye lids, eye brow area and under-eye area. Preferred vehicles therefore include vegetable or mineral oils, lotions, creams, milks, gels, aqueous liquids, emulsions, ointments, liniments, unguents, rubs, balms, salves, sera, mists, powders, liposomes and any other suitable topical formulation.

Preferred vehicles are water-soluble, have been clinically tested and do not cause eye or skin irritation. These vehicles and compositions made using them may be designed for application using the fingers or a suitable wand or swab, or may be provided in a container for application with a brush, wipe, swab, roller, spray, pen, pump or the like to deliver a precise or approximate amount of the product. The compositions may be applied in any convenient manner as discussed above. Most commonly, the composition is formulated in a liquid, semi-liquid, or gel formulation. In such cases, the composition is applied to the skin, for example the under eye area and gently blended into the skin with the fourth (ring) finger. Alternatively, the composition may be applied using an applicator device. The inventive compositions also may contain inert ingredients such as dyes and colorants, cosmetic tints or pigments to temporarily conceal dark circles, fragrances or flavorings, humectants, emollients, emulsifiers and surfactants, pH modifiers, binders, thickeners, and/or preservatives.

Although a primary use of the inventive composition is to reduce eye area puffiness and dark circles, the inventive compositions also can be used to treat inflammation and swelling on any area of the skin, wherever an anti-inflammatory action is desired, including other areas of the face or any body area, from any cause. For example, the compositions can be used on bruises, insect bites, allergic wheals, sunburn and the like, or wherever inflammation of the skin has occurred. In addition, the compositions can be applied to areas of the skin after cosmetic procedures such as laser treatments, electrolysis, waxing, peels (such as chemical peels) injections, piercings or tattoos to reduce swelling and inflammation and speed recovery.

Appropriate concentrations of the antihistamine compound for the inventive compositions range from about 0.0001% to about 99% or about 0.0001% to about 50% antihistamine compound by weight in a suitable vehicle. Preferably, the compositions contain about 0.001% to about 10% antihistamine or about 0.01% to about 5% antihistamine by weight. Most preferred compositions contain about 0.1% to about 3% or about 0.5% to about 2%, or about 1% antihistamine compound by weight. These percentage concentrations are approximately equivalent to 0.0001 mg antihistamine per cc of the composition to about 25 mg antihistamine per cc of the composition or about 0.001 to about 10, about 0.01 to about 5, about 0.1 to about 3, about 0.5 to about 2, or about 1 mg antihistamine per cc of the composition.

Inventive compositions also contain an NSAID. Such NSAIDs preferably are present in concentrations in the range from about 0.0001% to about 99% NSAID compound or about 0.0001% to about 50% NSAID by weight in a suitable vehicle. Preferably, the compositions contain about 0.001% to about 10% NSAID or about 0.01% to about 5% NSAID by weight. Most preferred compositions contain about 0.1% to about 3% or about 0.5% to about 2%, or about 1% NSAID compound by weight. The concentrations of the combined active agents (antihistamine and NSAID) therefore can range from 0.0002% to substantially 100%, although the preferable concentration is 1-2% for each. Useful percentage concentrations are approximately equivalent to 0.0001 mg NSAID per cc of the composition to about 25 mg NSAID per cc of the composition or about 0.001 to about 10, about 0.01 to about 5, about 0.1 to about 3, about 0.5 to about 2, or about 1 mg NSAID per cc of the composition. Desirable concentrations of steroid compounds are about 0.01% to about 50% by weight and preferably about 1% to about 10%, to provide a dosage per use of about 0.0001 mg to about 10 mg and preferably about 0.01 mg to about 0.1 mg of the steroid compound. These same ranges of concentration are suitable for leukotriene blocker compounds as well.

Preferred methods of using the product involve application to the skin of an amount of the composition of about 0.0001 cc to about 1 cc to an area of about 1-2 or 1-10 square inches, for example to each under eye or eye area of the skin, at least 1 or 2 times per month and up to 6 times per day. Any dosage schedule, such as once-a-week, once-a-day, or periodic use when the need arises is contemplated and within the scope of this invention. Preferably, the compositions described herein are applied to the skin about 2 times per day, using about 0.01 to about 1 cc or most preferably about 0.1 to about 0.2 ccs for each square inch of skin surface. Because the topical preparations of this invention are safe to use and non-irritating, the precise amount used is not critical. Therefore, dosage schemes outside these suggested ranges are contemplated for use.

In a study designed to test the effectiveness of the inventive compositions for amelioration of eye area darkness and swelling, a composition containing 1% fexofenadine and 1% Ibuprofen, dissolved in an inert vehicle, was applied topically to the area under the eyes of 59 patients. See Examples. This treatment resulted in a statistically significant improvement in appearance in all patients compared to vehicle alone. Furthermore, in other studies, the product containing 1% fexofenadine and 1% ibuprofen was more effective in reducing dark circles and lower eye swelling than either drug alone when applied topically. A third study found that topical application of an inventive composition (1% fexofenadine and 1% ibuprofen) resulted in statistically significant improvements in the appearance of dark circles and lower eye area swelling compared to systemic (oral) delivery of the two drugs in combination. Therefore, the inventive compositions have been shown to be more effective than comparable compositions delivered orally and more effective than either active component alone applied topically.

### EXAMPLES

### Example 1

### Double-Blind Placebo-Controlled Human Study of Topical Preparation

An embodiment of the inventive composition, referred to as the “test article” in this example, contained 1% 2-[4-(2-methylpropyl) phenyl] propanoic acid and fexofenadine in a vehicle of Kiehl's® brand Eye Alert®, a commercially available eye cream. The compositions were prepared aseptically. The test article and vehicle alone were placed in numbered, blinded syringes, with a safety cap in place.

In a double-blinded, placebo-controlled study in humans, 59 male and female patients, 18 years of age and older, received either test article (inventive composition) or vehicle alone (control) to apply topically to the under-eye area of the skin after informed consent was obtained. Each patient applied the test article to one side and the vehicle to the other, using a clean cotton-tipped applicator for each application per side, so that each patient could serve as her own control. The patients received two syringes, one with vehicle (control) and one containing the test article, with instructions about technique to apply the creams two times a day. Pre-treatment photos were obtained.

After one and two weeks, all patients completed a self-assessment. A blinded examiner then rated the patients in terms of clinical improvement and differences between the two sides. The amount of test article remaining was weighed and returned at the end of Week 1 and weighed and collected at the end of Week 2. The patients returned again after one week off-therapy (the end of Week 3) to be photographed and to complete a self-assessment. The same blinded examiner scored the patients in terms of clinical results.

Patients were treated for 4 weeks, twice a day (through Visits 1-5) with active drug to one side of the face and placebo (vehicle alone) to the other side of the face. The patient and the investigator were blinded as to which side was treated with which formulation. At weekly visits on therapy (Visits 2-5) and two weeks off therapy (Visit 6), both the patients themselves and the Investigator rated the patients in terms of clinical improvement or worsening and differences between the two sides. Patients were photographed at each visit. The test articles were weighed to assure compliance.

All patients reported a significant improvement in lower eye area swelling and darkness on the test article side versus the vehicle alone side. The examiner agreed with these assessments. There were no adverse events.

### Example 2

### Effectiveness of Oral Combination Therapy Compared to Topical Combination Therapy in Humans

After informed consents were obtained, seven women, age 18 or older, received oral medication, 100 mg Ibuprofen and 60 mg Fexofenadine, to be taken twice daily for one week. The medications were delivered in a blister pack to lessen the chance of dosing error and to increase compliance. The blister packs for the oral medication were returned at the end of Week 1. At that time, the women received a topical treatment composition containing 1% Fexofenadine and 1% Ibuprofen, dissolved in a stable and commercially available eye cream, to be applied to each side under the eyes, twice a day, using a clean cotton tipped applicator. Each applicator was to be used once only and for only one side. The patients returned the topical treatment composition at the end of Week 2 for weighing of the test article. Photographs of the peri-orbital areas of all patients were taken at entry, end of Week 1, and end of Week 2. The oral combination fexofenadine and ibuprofen caused no improvement in the lower eye area swelling or darkness, but there was a significant improvement in the appearance of swelling and darkness in all patients as early as Day 3 after beginning topical therapy.

### Example 3

### Effectiveness of Topical Single-Drug Therapy Compared to Topical Combination Therapy in Humans

After informed consent was obtained, six female patients, age 18 and older, received a composition according to an embodiment of the invention containing 1% Fexofenadine and 1% Ibuprofen in an inert eye cream and control compositions containing vehicle plus 1% Fexofenadine or vehicle plus 1% Ibuprofen. The patients were instructed to apply the products with a clean cotton tipped applicator topically to the under eye area, each on one side of the face, with a separate clean cotton tipped applicator.

During Week 1, the patients applied the combination product to one side and either fexofenadine alone or ibuprofen alone (chosen at random for the week) to the other side. During Week 2, treatment with the combination product was maintained on the same side of the face, while the other side was switched from one compound alone to the other (i.e., from fexofenadine to ibuprofen or from ibuprofen to fexofenadine). Photographs and patient satisfaction surveys were administered at entry, at the end of Week 1, and at the end of Week 2. Topical antihistamine alone and topical ibuprofen alone did not improve lower eye swelling and darkness, but the combination 1% fexofenadine and 1% ibuprofen according to an embodiment of the invention caused a significant improvement in lower eye swelling and darkness.

1. A topical composition which comprises an antihistamine compound, a non-steroidal anti-inflammatory drug (NSAID) compound and a pharmaceutically acceptable vehicle for topical administration.

2. The topical composition of claim 1, wherein said antihistamine compound is selected from the group consisting of fexofenadine, loratadine, desloratadine, azelastine, cetirizine and levocetirizine.

3. The topical composition of claim 1, wherein said antihistamine compound is fexofenadine.

3. The topical composition of claim 1, which contains about 0.0001% to about 99% of said antihistamine compound by weight.

4. The topical composition of claim 1, which contains about 0.0001% to about 50% of said antihistamine compound by weight.

5. The topical composition of claim 1, which contains about 0.001% to about 10% of said antihistamine compound by weight.

6. The topical composition of claim 1, which contains about 0.01% to about 5% of said antihistamine compound by weight.

7. The topical composition of claim 1, which contains about 0.1% to about 3% of said antihistamine compound by weight.

8. The topical composition of claim 1, which contains about 0.5% to about 2% of said antihistamine compound by weight.

9. The topical composition of claim 1, which contains about 1% of said antihistamine compound by weight.

10. The topical composition of claim 1, wherein said NSAID compound is selected from the group consisting of ibuprofen, aspirin, ampyrone, celecoxib, diclofenac, diflunisal, droxcam, indomethacin, licofelone, mefanamic acid, naproxen, nimesulide, phenylbutazone, proicam, rofecoxib, valdecoxib, omega-3 fatty acids, and any combination thereof.

11. The topical composition of claim 10, wherein said NSAID compound is ibuprofen.

12. The topical composition of claim 1, which contains about 0.0001% to about 99% of said NSAID compound by weight.

13. The topical composition of claim 1, which contains about 0.0001% to about 50% of said NSAID compound by weight.

14. The topical composition of claim 1, which contains about 0.001% to about 10% of said NSAID compound by weight.

15. The topical composition of claim 1, which contains about 0.01% to about 5% of said NSAID compound by weight.

16. The topical composition of claim 1, which contains about 0.1% to about 3% of said NSAID compound by weight.

17. The topical composition of claim 1, which contains about 0.5% to about 2% of said NSAID compound by weight.

18. The topical composition of claim 1, which contains about 1% of said NSAID compound by weight.

19. The topical composition of claim 1, wherein said antihistamine compound is fexofenadine and wherein said NSAID compound is ibuprofen.

20. The method of treating swelling, puffiness, redness, darkness or inflammation of skin of a human in need thereof, which comprises topically applying to said skin the topical composition of claim 1.

21. The method of treating swelling, puffiness, redness, darkness or inflammation of skin of a human in need thereof, which comprises topically applying to said skin the topical composition of claim 19.

22. The method of claim 20, wherein said skin is the eye area.

23. The method of claim 21, wherein said skin is the eye area.

24. A method of treating darkness of the skin under or around the eye of a human in need thereof, which comprises topically applying to said skin the topical composition of claim 1.
